(19) United States
(12) Patent Application Publication
(10) Pub. No.: US 2011/0129846 A1
(43) Pub. Date: Jun. 2, 2011

(54) PHOTONIC BIOSENSOR, PHOTONIC BIOSENSOR ARRAY, AND METHOD OF DETECTING BIOMATERIALS USING THE SAME

(75) Inventors: Chul Huh, Daejeon (KR); Kyung Hyun Kim, Daejeon (KR); Bong Kyu Kim, Daejeon (KR); Hyun Sung Ko, Seoul (KR); Wan Joong Kim, Goyang (KR); Jong Cheol Hong, Daejeon (KR); Gun Yong Sung, Daejeon (KR); Seon Hee Park, Daejeon (KR)

(73) Assignee: Electronics and Telecommunications Research Institute, Daejeon (KR)

(21) Appl. No.: 12/860,796

(22) Filed: Aug. 20, 2010

(30) Foreign Application Priority Data
Nov. 27, 2009 (KR) 10-2009-0115976

(51) Int. Cl. G01N 33/53; G01N 33/543; C12M 1/34; G01N 30/96

(52) U.S. Cl. 435/7.1; 436/518; 435/287.2; 422/69

(57) ABSTRACT

A photonic biosensor, a photonic biosensor array, and a method of detecting a bio-material using the same are provided. The photonic biosensor includes a light emitting diode configured to emit light, a photodiode (PD), an optical fiber configured to connect the light emitting diode with the PD, and a micro-fluidic channel disposed on the optical fiber. Bio-antibodies or aptamers are fixed to the surface of the optical fiber, and the micro-fluidic channel includes gold (Au) nanoparticles to which bio-antibodies or aptamers are fixed. The photonic biosensor may be configured using absorption of surface plasmons in Au nanoparticles with respect to light traveling through the surface of the optical fiber configured to connect the light emitting diode with the PD, thus simplifying the manufacture of the biosensor and reducing the manufacturing cost.

### CROSS-REFERENCE TO RELATED APPLICATION

This application claims priority to and the benefit of Korean Patent Application No. 10-2009-0115976, filed Nov. 27, 2009, the disclosure of which is incorporated herein by reference in its entirety.

### BACKGROUND

1. Field of the Invention

The present invention relates to a photonic biosensor, a photonic biosensor array, and a method of detecting a biomaterial using the same, and more specifically, to a photonic biosensor, a photonic biosensor array, and a method of detecting biomaterials using the same, which employ absorption of surface plasmon in gold (Au) nanoparticles with respect to light traveling through the surface of an optical fiber.

2. Discussion of Related Art

A biosensor may include a bio-sensing material and a signal detector and may be capable of selectively detecting a material to be analyzed. The bio-sensing material may be an enzyme, an antibody, or deoxyribonucleic acid (DNA), which may selectively react and bond with a specific material. Also, the signal detector may detect a signal of a bio-material using various physicochemical methods. For example, the signal detector may detect the signal by measuring a minute electrical variation (e.g., voltage, current, or resistance) according to the presence or absence of the bio-material, a variation in fluorescent intensity due to a chemical reaction, or a variation in an optical spectrum.

The above-described biosensor may be applied not only to genetic research but also to medical purposes, such as initial diagnosis of diseases and management of chronic diseases, and to biosensors for the environment, foodstuffs, and military and industrial purposes. In general, the diagnosis of diseases and the management of the chronic diseases may broadly employ a testing method based on color formation due to a chemical reaction of an enzyme or a method of measuring sensitivity, such as fluorescent intensity.

Furthermore, with developments in research on antibodies or aptamers that uniquely bond with specific bio-materials, a considerable amount of research has focused on methods of detecting bio-materials by means of immunoassay with high sensitivity, precision, and reliability using the unique bonding of the antibodies or aptamers with the specific bio-materials. A conventional method of detecting a bio-material may be typically performed using a label biosensor. Thus, the conventional method of detecting the bio-material may include labeling a specific antibody with a radioactive isotope or fluorescent material, allowing the corresponding antigen to react with the specific antibody, and quantitatively measuring a specific antigen due to a variation in radiation or a variation in fluorescent intensity. The above-described method involves an additional process of labeling a specific antibody with a fluorescent material expressing specific color, thus complicating the entire process and increasing the process cost.

Therefore, a vast amount of research has lately been conducted on photonic biosensors as label-free biosensors, which are free from a label material, such as a fluorescent material expressing a specific color. The photonic biosensors may include surface plasma biosensors, total internal reflection ellipsometry biosensors, and waveguide biosensors.

Each of the photonic biosensors, such as the surface plasma biosensors, the total internal reflection ellipsometry biosensors, and the wavelength biosensors, may include a light source configured to emit light, a reaction unit where an antibody-antigen reaction occurs, and a detector configured to measure a photo-signal. The light source configured to emit light may be a light emitting diode (LED) or an emission laser. Also, the detector configured to detect a variation in photo-signal may typically be a spectrometer.

A typical photonic biosensor may include a light source configured to emit light and a detector (i.e., spectrometer) configured to detect a variation in photo-signal. In this case, the photo-signal output by the detector may be varied very sensitively according to a direction in which light is incident from the light source to a reaction unit where a reaction of a specific antibody with an antigen occurs. Also, to measure a light spectrum, the light source configured to emit light should be a wavelength-varying light source or the detector configured to detect the variation in photo-signal should be used as the spectrometer. Accordingly, a very complicated optical system may be required to configure the light source and the detector, thus increasing the fabrication cost of the biosensor.

### SUMMARY OF THE INVENTION

The present invention is directed to a photonic biosensor, which may measure a variation in photocurrent of light traveling through the surface of an optical fiber due to a reaction of bio-antibodies or aptamers adsorbed onto the surface of the optical fiber with gold (Au) nanoparticles to facilitate detection of a bio-material.

Also, the present invention is directed to a method of detecting a bio-material using a photonic biosensor including an optical fiber, a light emitting diode, and a photodiode (PD).

One aspect of the present invention provides a photonic biosensor including: a light emitting diode configured to emit light; a PD; an optical fiber configured to connect the light emitting diode with the PD; and a micro-fluidic channel disposed on the optical fiber, wherein a bio-antibody or aptamer is fixed to the surface of the optical fiber, and the micro-fluidic channel includes Au nanoparticles to which bio-antibodies or aptamers are fixed.

The light emitting diode may have a single light source. The PD may have the highest sensitivity to the single light source of the light emitting diode.

The optical fiber may have a length of about 1 nm to 10 cm.

The micro-fluidic channel may be formed using silicon (PDMS) on the optical fiber. The bio-antibodies or aptamers may be fixed to the surface of the optical fiber using physical adsorption or chemical bonding. Bio-antibodies or aptamers may be fixed to Au nanoparticles included in the micro-fluidic channel using physical adsorption or chemical bonding.

Another aspect of the present invention is to provide a photonic biosensor array including a plurality of photonic biosensors, each photonic biosensor according to the present invention.

Another aspect of the present invention provides a method of detecting a biomaterial using the photonic biosensor including: a light emitting diode configured to emit light; a PD; an optical fiber configured to connect the light emitting diode with the PD; and a micro-fluidic channel disposed on the optical fiber, wherein bio-antibodies or aptamers are fixed to the surface of the optical fiber, and the micro-fluidic channel includes Au nanoparticles to which bio-antibodies or aptamers are fixed. The method includes: measuring a photocurrent of the photonic biosensor; allowing a solution containing bio-antigens to be detected to flow through a micro-fluidic channel of the photonic biosensor; bonding the bio-antigens between bio-antibodies or aptamers fixed to the surface of an optical fiber of the photonic biosensor and bio-antibodies or aptamers fixed to Au nanoparticles; and measuring a photocurrent of the photonic biosensor after the bonding of the antigens to detect specific bio-antigens.

The Au nanoparticles to which the bio-antibodies or aptamers are fixed and the solution containing the bio-antigens may be sequentially or simultaneously allowed to flow through the micro-fluidic channel.

The detection of the specific bio-antigens may include comparing a photocurrent measured by the PD before a reaction of the bio-antibodies or aptamers with the bio-antigens with a photocurrent measured by the PD after the reaction.

The solution containing the bio-antigen may be blood, urine, or saliva.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a schematic diagram of a structure of a photonic biosensor according to an exemplary embodiment of the present invention;

FIG. 2 is a schematic diagram of a photonic biosensor array including a plurality of photonic biosensors according to an exemplary embodiment of the present invention;

FIG. 3 is a flowchart illustrating a process of detecting a biomaterial using a photonic biosensor according to an exemplary embodiment of the present invention;

FIG. 4 is a schematic diagram of a process of detecting a biomaterial using a photonic biosensor according to an exemplary embodiment of the present invention; and

FIG. 5 is a graph showing a photocurrent output by a photodiode (PD) using a photonic biosensor according to an exemplary embodiment of the present invention before and after a reaction of bio-, biological, biochemical, or environmental antibodies with an antigen.

### DETAILED DESCRIPTION OF EXEMPLARY EMBODIMENTS

The present invention will now be described more fully hereinafter with reference to the accompanying drawings, in which exemplary embodiments of the invention are shown. Descriptions of well-known components and processing techniques are omitted so as not to unnecessarily obscure the embodiments of the present invention. Like numbers refer to like elements throughout.

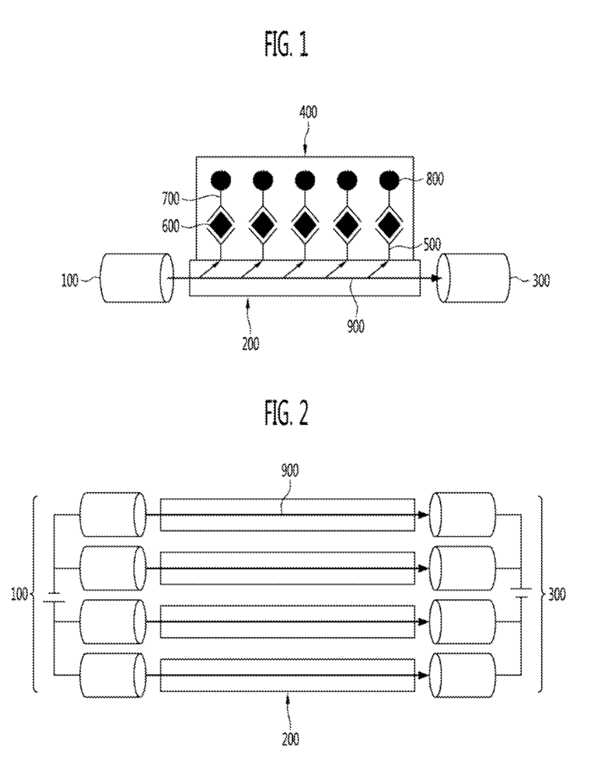

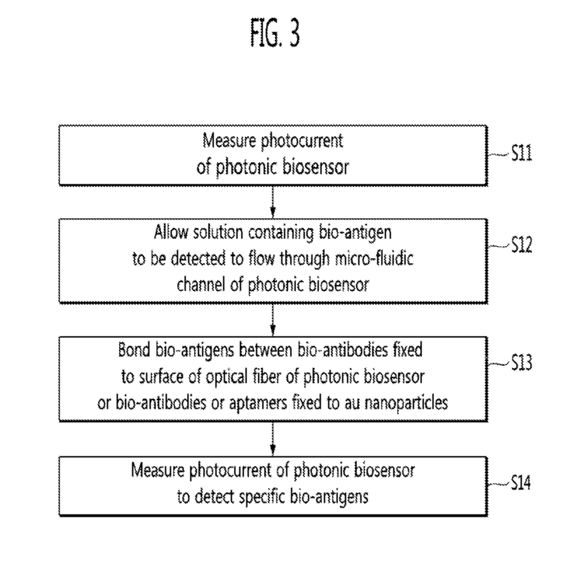

FIG. 1 is a schematic diagram of a structure of a photonic biosensor according to an exemplary embodiment of the present invention, and FIG. 2 is a schematic diagram of a photonic biosensor array including a plurality of photonic biosensors according to an exemplary embodiment of the present invention.

Referring to FIG. 1, a photonic biosensor according to an exemplary embodiment of the present invention may include a light emitting diode 100, a photodiode (PD) 300, an optical fiber 200 configured to connect the light emitting diode 100 with the PD 300, and a micro-fluidic channel 400 disposed on the optical fiber 200. Bio-antibodies or aptamers 500 may be fixed to the surface of the optical fiber 200, and the micro-fluidic channel 400 may include gold (Au) nanoparticles 800 to which bio-antibodies or aptamers 700 are fixed.

The light emitting diode 100 may be a light emitting diode having a single light source configured to emit light and vary the wavelength of light according to the size of the Au nanoparticles 800.

The PD 300, which may be a sensor configured to measure a variation in photocurrent, may be a PD having the highest sensitivity in the wavelength range of the light emitting diode having the single light source.

A micro-fluidic channel 400, which is a path through which light 900 travels from the light emitting diode 100 to the PD 300, may be formed on the optical fiber 200, and bio-antibodies or aptamers 500 may be fixed to the surface of the optical fiber 200.

Here, the optical fiber 200 may be an ordinary optical fiber, which may have a length of about 1 nm to 10 cm.

The micro-fluidic channel 400 may be formed on the optical fiber 200 using silicon (PDMS). The micro-fluidic channel 400 may have a length of about 1 nm to 50 cm and a capacity of about 1 nl to 1 ml. The bio-antibodies or aptamers 700 may be fixed to the Au nanoparticles 800 included in the micro-fluidic channel 400 using a biological or physicochemical method.

The Au nanoparticles 800 have intrinsic light absorptivity. Thus, when the light 900 travels from the light emitting diode 100 through the optical fiber 200, the Au nanoparticles 800 may absorb the light 900. Accordingly, the PD 300 may sense specific bio-molecules by measuring the photocurrent, and quantitatively detect the specific bio-molecules based on a variation in the photocurrent.

A plurality of photonic biosensors according to the present invention may constitute a photonic biosensor array shown in FIG. 2. Thus, a sensor system capable of easily detecting various bio-materials at once may be configured using the photonic biosensor array.

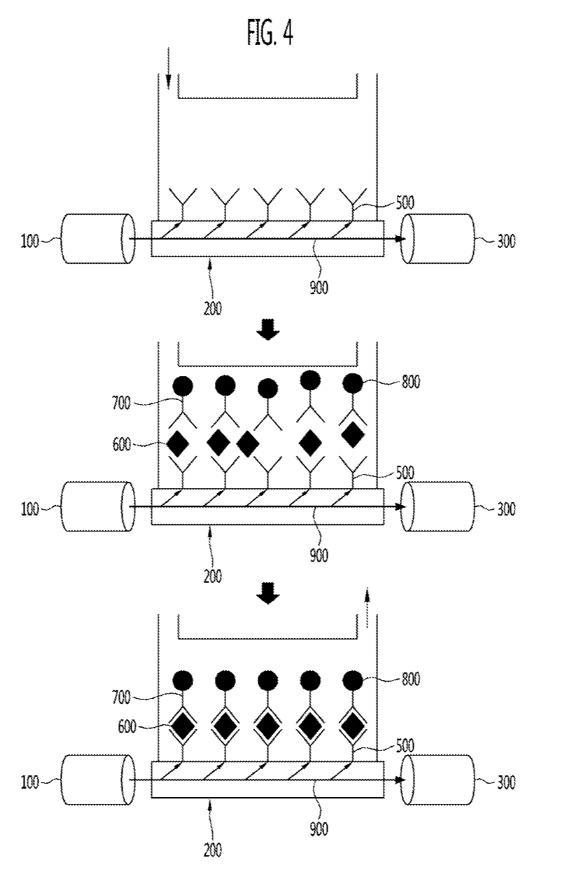

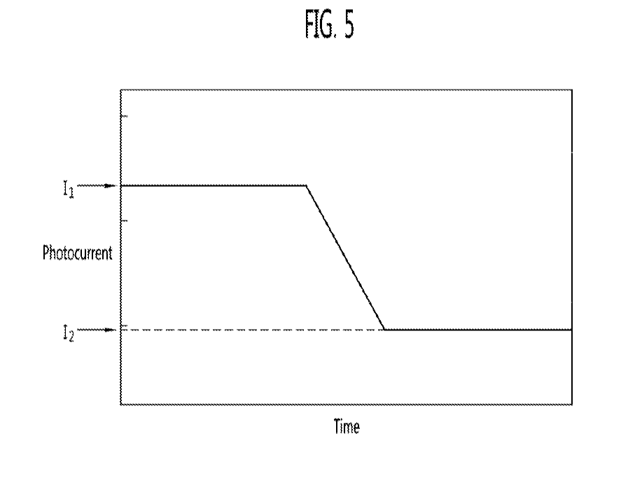

FIG. 3 is a flowchart illustrating a process of detecting a biomaterial using a photonic biosensor according to an exemplary embodiment of the present invention, and FIG. 4 is a schematic diagram of a process of detecting a biomaterial using a photonic biosensor according to an exemplary embodiment of the present invention.

Referring to FIGS. 3 and 4, a method of detecting a biomaterial using a photonic biosensor according to an exemplary embodiment of the present invention may include: measuring a photocurrent of the photonic biosensor (operation S11); allowing a solution containing bio-antigens 600 to be detected to flow through a micro-fluidic channel 400 of the photonic biosensor (operation S12); bonding the bio-antigens 600 between bio-antibodies or aptamers 500 fixed to the surface of the optical fiber 200 and bio-antibodies or aptamers 700 fixed to Au nanoparticles 800 (operation S13); and measuring the photocurrent of the photonic biosensor after bonding the bio-antigens 600 to detect specific bio-antigens (operation S14).

In operation S11, the photocurrent may be measured before the bio-antigens 600 are bonded. Thus, the photocurrent measured before the bio-antigens 600 are bonded may be compared with the photocurrent measured after the bio-antigens 600 are bonded.

In operation S12, the Au nanoparticles 800 to which the bio-antibodies or aptamers 700 are fixed and a solution containing the bio-antigens 600 may be allowed to flow together through the micro-fluidic channel 400 of the photonic biosensor. The solution containing the bio-antigens 600 may be, for example, blood, urine, or saliva. That is, the solution may be injected through an inlet port of the micro-fluidic channel 400 and exhausted through an outlet port thereof. In this case, the solution containing the bio-antigens 600 may be allowed to flow at a rate of about 1 nl/min to 1 ml/min.

In operation S13, the bio-antigens 600 contained in the solution may be bonded between the bio-antibodies or aptamers 500 fixed to the surface of the optical fiber 200 and the bio-antibodies or aptamers 700 fixed to the Au nanoparticles 800.

In operation S14, a photocurrent obtained by absorbing light 900 traveling from the light emitting diode 100 to the PD 300 after the bio-antigens 600 are bonded may be measured and compared with the photocurrent measured before the bio-antigens 600 are bonded. Thus, the concentration of antigens in the solution may be analyzed based on a difference in photocurrent.

FIG. 5 is a graph showing a photocurrent output by a PD using a photonic biosensor according to an exemplary embodiment of the present invention before and after a reaction of bio-, biological, biochemical, or environmental antibodies with antigens.

In FIG. 5, a variation in photocurrent output by a PD due to a reaction caused between bio-materials on the surface of an optical fiber 200 is illustrated with a dotted line. After a reaction of antigens 600 included in a solution (e.g., blood, urine, or saliva) with antibodies 500 fixed to the surface of the optical fiber or antibodies fixed to the surface of Au nanoparticles in a micro-fluidic channel, a difference (I1-I2) in photocurrent, which may be caused by intrinsic absorption of the Au nanoparticles, may be measured to obtain the concentration of the antigens. Also, as the concentration of the antigens to be detected in the micro-fluidic channel 900 in the solution (e.g., blood, urine, or saliva) increases, the difference (I1-I2) between the photocurrents measured before and after the antigen-antibody reaction may further increase. Therefore, by measuring the difference in the photocurrent, the concentration of the antigens in the solution may be analyzed very precisely, sensitively, and quantitatively.

A photonic biosensor using absorption of surface plasmon in Au nanoparticles with respect to light traveling through the surface of an optical fiber may be configured with a light source, an optical fiber, and a PD, thereby facilitating the manufacture of the biosensor and reducing the fabrication costs.

Furthermore, since a light emitting diode functioning as a light source, an optical fiber functioning as a reaction unit, and a PD configured to measure a photocurrent of light traveling through the optical fiber and the reaction unit are small-sized, an optical measurement system may be simply manufactured, and a sensor array capable of measuring various bio-materials may be easily configured.

A photonic biosensor according to an exemplary embodiment may be employed to quantitatively sense and detect a bio-, biological, biochemical, or environmental material included in a solution, such as blood, urine, saliva, and the like.

Although exemplary embodiments of the present invention have been described with reference to the attached drawings, the present invention is not limited to these embodiments, and it should be appreciated to those skilled in the art that a variety of modifications and changes can be made without departing from the spirit and scope of the present invention.

What is claimed is:

1. A photonic biosensor comprising:
a light emitting diode configured to emit light;
a photodiode (PD);
an optical fiber configured to connect the light emitting diode with the PD; and
a micro-fluidic channel disposed on the optical fiber,
wherein bio-antibodies or aptamers are fixed to the surface of the optical fiber, and the micro-fluidic channel includes gold (Au) nanoparticles to which bio-antibodies or aptamers are fixed.

2. The photonic biosensor of claim 1, wherein the light emitting diode has a single light source.

3. The photonic biosensor of claim 1, wherein the PD has the highest sensitivity to the single light source of the light emitting diode.

4. The photonic biosensor of claim 1, wherein the optical fiber has a length of about 1 nm to 10 cm.

5. The photonic biosensor of claim 1, wherein the micro-fluidic channel is formed using silicon (PDMS) on the optical fiber.

6. The photonic biosensor of claim 1, wherein the bio-antibodies or aptamers are fixed to the surface of the optical fiber using physical adsorption or chemical bonding.

7. The photonic biosensor of claim 1, wherein the bio-antibodies or aptamers are fixed to Au nanoparticles included in the micro-fluidic channel using physical adsorption or chemical bonding.

8. A photonic biosensor array comprising:
a plurality of light emitting diodes configured to emit light;
a plurality of PDs;
a plurality of optical fibers configured to connect the plurality of light emitting diodes with the PDs; and
a micro-fluidic channel disposed on each of the plurality of optical fibers,
wherein bio-antibodies or aptamers are fixed to the surface of each of the optical fibers, and each of the micro-fluidic channels includes gold (Au) nanoparticles to which bio-antibodies or aptamers are fixed.

9. A method of detecting a biomaterial using a photonic biosensor, the method comprising:
measuring a photocurrent of the photonic biosensor according to claim 1;
allowing a solution containing bio-antigens to be detected to flow through a micro-fluidic channel of the photonic biosensor;
bonding the bio-antigens between bio-antibodies or aptamers fixed to the surface of an optical fiber of the photonic biosensor and bio-antibodies or aptamers fixed to Au nanoparticles; and
measuring a photocurrent of the photonic biosensor after bonding the bio-antigens to detect specific bio-antigens.

10. The method of claim 9, wherein the Au nanoparticles to which the bio-antibodies or aptamers are fixed and the solution containing the bio-antigens are sequentially or simultaneously allowed to flow through the micro-fluidic channel.

11. The method of claim 9, wherein detecting the specific bio-antigens comprises comparing a photocurrent measured by the PD before a reaction of the bio-antibodies or aptamers with the bio-antigens with a photocurrent measured by the PD after the reaction.

12. The method of claim 9, wherein the solution containing the bio-antigens is blood, urine, or saliva.
